(19) United States
(12) Patent Application Publication
(10) Pub. No.: US 2015/0257702 A1
(43) Pub. Date: Sep. 17, 2015

(54) Insulation Tube for an Electric Line for Medical Use, and Method for Producing Such a Tube

(71) Applicant: BIOTRONIK SE & Co. KG, Berlin (DE)

(72) Inventors: Pierre Weitzig, Irrel (DE); Detmar Jadwizak, Erkner (DE); Gordon Hillebrand, Berlin (DE); Carsten Fruendt, Berlin (DE)

(21) Appl. No.: 14/628,743

(22) Filed: Feb. 23, 2015

Related U.S. Application Data
(60) Provisional application No. 61/950,846, filed on Mar. 11, 2014.

(51) Int. Cl. A61B 5/00; A61N 1/05; B29C 63/22; H01B 7/24

(52) U.S. Cl. CPC A61B 5/6847; H01B 7/24; A61N 1/05; B29C 63/22; A61B 2562/182; A61B 2562/222

(57) ABSTRACT

An insulation tube for an electric line for medical use, in particular a cardiac pacemaker electrode line, defibrillator electrode line, electric line for nerve stimulation, electric line in catheters, or the like, which, in order to better transmit tensile forces, includes a matrix consisting of resilient material and web-like elements consisting of rigid plastic material and embedded in the matrix, wherein the web-like elements run at least in portions along a helix line which forms an angle of at least 10° in relation to the longitudinal axis of the insulation tube. The invention further relates to a method for producing an insulation tube of this type and also to an electric line including comprising such an insulation tube.

### CROSS-REFERENCE TO RELATED APPLICATIONS

This patent application claims the benefit of co-pending U.S. Provisional Patent Application No. 61/950,846, filed on Mar. 11, 2014, which is hereby incorporated by reference in its entirety.

### TECHNICAL FIELD

The present invention relates to an insulation tube for an electric line for medical use, in particular a cardiac pacemaker electrode line, a defibrillator electrode line, an electrode line for nerve stimulation, an electric line in catheters, and the like, consisting of a resilient material. The present invention further relates to an electric line of this type and also to a method for producing an insulation tube for an electric line of this type.

### BACKGROUND

Electronic medical devices, such as, for example, cardiac pacemakers (pacemakers), defibrillators or neurological devices, such as, for example, brain pacemakers for deep brain stimulation, spinal cord stimulation devices, TENS (transcutaneous electrical nerve stimulator), ablation and diagnostic devices (for catheters), or devices for muscular stimulation therapy and diagnosis devices which examine the chemical properties of the blood of the patient, of other body parts, or other body properties and parameters, often use electric lines that are guided into the body of the patient, where they remain at least for the duration of the treatment or measurement. The electric lines are electrically conductively connected to the electronic device, which may be implantable, where applicable.

These medical devices usually comprise a housing, compatible with the body, where applicable, having an associated electronic circuit and a power supply, for example, a battery. The housing has at least one connection point, to which the electric line or the electric lines can be connected. The electric line or the electric lines serve to transmit the electric energy or signals from the housing to the treated or examined body part, and vice versa.

Here, the term “electric line” in the field of medical technology not only denotes an element with which electric energy is transmitted in accordance with the physical definition, but includes a line having an electric conductor together with its enveloping insulation, which is often formed as an insulation tube, and also all further functional elements fixedly connected to the line. For example, the electric line also comprises what is known as the electrode tip, by means of which the electric energy is introduced into the tissue to be treated. An electrode tip is often also provided with anchor elements or retaining structures, with which the stability of the spatial position of the point of transmission of the electric energy in the tissue to be treated is ensured. The electrode tip, which forms a point of transmission of the electric energy into the tissue, can be designed as a recording electrode, a stimulation electrode or a measuring electrode.

With electric lines of this type, an insulation tube made of silicone is often used to insulate the electrically conductive elements outside the connection point and the electrode tip. Silicones have a high biocompatibility, long-term stability, a sufficient hardness and also a high resilience.

Due to the high resilience and the low wall thickness usually used for such an insulation tube, an insulation tube made of silicone can only take up low forces however, in particular tensile forces, and further in particular in the region of a ring electrode or the tip of the electric line, which is often formed as an electrode tip. The reliable transmission of tensile force from one side to the other side of the ring electrode and the reliable anchoring of the electrode tip are thus impaired.

It is therefore desirable to increase the proportion of tensile forces that can be transmitted via the insulation tube with constant wall thickness, wherein the inflatability of the insulation tube is to be maintained in order to facilitate the assembly thereof.

A catheter for medical procedures is known from document U.S. Publication No. 2011/0054312, of which the shaft has a reinforced portion. Wire-like elements running in the longitudinal direction of the shaft can be introduced for reinforcement. However, this reinforcement is still insufficient for many applications.

A tube for a medical device containing a fiber-reinforced polymer is described in document U.S. Publication No. 2012/0310180. However, this polymer is arranged in an inner layer of the tube and means that this tube cannot be inflated.

The present invention is directed toward overcoming one or more of the above-mentioned problems.

### SUMMARY

An object is therefore to create an insulation tube and also a corresponding electric line which can transmit high tensile forces with low wall thickness. Here, an insulation tube of this type is also to be extendible in the radial direction in order to enable the assembly of the tube over larger component parts. An object accordingly also consists in specifying a method for producing an insulation tube of this type, said method being cost-effective and easily executable.

At least the above objects are achieved by an insulation tube having the features of claim 1, by an electric line having the features of claim 13, and also by the methods for producing an insulation tube having the features of claims 13 and 15.

In particular, the insulation tube according to the present invention comprises a matrix consisting of resilient material and web-like elements embedded in the matrix, said elements consisting of rigid plastic material, wherein the web-like elements run at least in portions along a helix line which forms an angle of at least 10° in relation to the longitudinal axis of the insulation tube. The angle that the web-like elements form in portions with the longitudinal axis of the insulation tube is preferably at most 30°.

The above-specified insulation tube according to the present invention comprising the web-like reinforcing elements running along a helix line has the advantage that it can transmit high tensile forces with a low wall thickness since the insulation tube is not extendible in the axial direction, that is to say in the direction of the longitudinal axis, due to the rigid web-like elements. The resilient part of the insulation tube according to the present invention can also expand in the radial direction. This radial extension results in an axial shortening of the insulation tube, if only to a small extent. The helical twisting of the web-like elements produces, in the event of axial tensile loading, an anchoring of these elements in the resilient part of the insulation tube, such that a displacement of the rigid elements with respect to the resilient matrix is prevented.

The extensibility of the insulation tube in the radial direction is also enabled, in particular since the web-like elements are provided separately from one another in the insulation tube, in particular distributed over the entire periphery of the insulation tube, such that an individual web-like element extends only over part of the periphery and does not encompass the entire periphery of the insulation tube. The insulation tube can expand in accordance with the present invention in such a way that the tube can be drawn over other component parts under radial stress, for example, during the assembly process.

An insulation tube of this type is formed particularly easily if a number of web-like elements extend over the entire axial length of the insulation tube. In this case, the insulation tube according to the present invention has the above-specified properties along its entire axial length.

In order to achieve a particularly good anchoring of the web-like elements in the matrix formed from resilient material, apertures, that is to say openings, which extend over the entire thickness of the corresponding web-like element, can be provided in the web-like elements. The matrix formed of resilient material flows into these openings in the web-like elements during the production of an insulation tube according to the present invention.

A good anchoring in the resilient matrix is likewise achieved if the web-like elements are formed in an S-shaped manner.

In a preferred exemplary embodiment, the web-like elements have a thickness (dimension in the radial direction) of 0.15 mm at most, preferably of 0.1 mm at most, and particularly preferably of 0.03 mm at most. Such a thickness of the web-like elements is sufficient in order to produce the necessary stability for the insulation tube.

The web-like elements preferably comprise a rigid thermoplastic material, and preferably a compound from the group containing polyimide, polyurethane, polyamide, PTFE (polytetrafluoroethylene). It is also advantageous if the matrix consists of a resilient plastic and preferably comprises a compound from the group containing silicones, rubber and thermoplastic elastomers (TPEs), for example, a polyether block amide.

At least the above objects are also achieved by an electric line comprising an above-described insulation tube, wherein the insulation tube is preferably arranged in the region of a ring electrode and/or an electrode tip, wherein an arrangement along the electric line over a large part of the length thereof or over the entire length thereof is also possible. The electric line preferably comprises an inflatable balloon. It is further preferable if the above-described insulation tube is arranged between an inner electrode feed line (coil) and the ring electrode and/or the electrode, preferably at the electrode tip, and surrounds the electrode feed line in the region of the respective electrode. Besides the respective electrode, a further insulating sleeve, which, for example, consists of silicone or has the structure of the insulation tube according to the present invention, can be provided above the above-described insulation tube.

The line according to the present invention has the advantages described above in conjunction with the insulation tube. In the case of the use of an insulation tube according to the present invention on a heel/shoulder of an electrode tip or a ring electrode of an electric line, an improved anchoring of such a reinforced insulation tube is achieved since a very resilient material (for example, silicone) is adhesively bonded onto a fixed/rigid substrate in the conventional manner. In the event of a tensile loading, the conventional structure results in a flaking due to stretching. If the insulation tube according to the present invention is used, a stretching and consequently a flaking of the electrode are prevented here.

A cost-effective and simple production method for such an insulation tube comprises the following steps:
manufacturing a first thin-walled tubing from a rigid plastic material, preferably a rigid thermoplastic material,
introducing openings into the rigid plastic material over part of the length in a central portion of the first tubing, wherein the openings define web-like elements in the tube in such a way that the web-like elements run at least in portions along a helix line which forms an angle of at least 10° in relation to the longitudinal axis of the tubing,
embedding the first tubing in a matrix consisting of a resilient material, such that a second tubing is formed which is composed of the first tubing and the surrounding matrix,
cutting off the ends of the second tubing in such a way that, of the first tubing, merely at least part of the central portion remains and forms the insulation tube.

The production method is designed in a particularly simple manner if, in order to embed the first tube in the matrix formed of the resilient material, the first tube is over-molded with the material of the matrix. For this purpose, the first tube is preferably placed with an inlaid core wire in an injection mold.

Alternatively, the insulation tube according to the present invention can be produced by means of extrusion and, more specifically, a tubing is first formed from the resilient material by means of extrusion and the web-like elements are then embedded in the extruded material of the matrix, which is not yet fully cured.

If necessary, the intermediate product thus produced is then twisted in a further exemplary embodiment, where applicable with additional heat supply, in such a way that the web-like elements run at least in portions along a helix line which forms an angle of at least 10° in relation to the longitudinal axis of the insulation tube.

Further objectives, aspects, features, advantages and possibilities for use of the present invention will emerge from a study of the following description of exemplary embodiments of the present invention with reference to the figures, and also the appended claims. Here, all described features and/or features presented in images form the subject matter of the present invention, either individually or in any combination, irrespective of their summary in the individual claims or the dependency references of the claims.

### DESCRIPTION OF THE DRAWINGS

In the drawings:

FIG. 1 schematically shows a perspective view from the side of a first exemplary embodiment of an insulation tube according to the invention,

FIG. 1A schematically shows a view from the side of the insulation tube according to FIG. 1,

FIG. 2 schematically shows a perspective view from the side of a first step of the production method for the insulation tube according to FIG. 1,

FIG. 3 schematically shows a perspective view from the side of a second step of the production method for the insulation tube according to FIG. 1,

FIG. 4 schematically shows a perspective view from the side of a second exemplary embodiment of an insulation tube according to the present invention,

FIG. 5 schematically shows a perspective view from the side of a third exemplary embodiment of an insulation tube according to the present invention,

FIG. 6 schematically shows a perspective view from the side of a fourth exemplary embodiment of an insulation tube according to the present invention,

FIG. 7 schematically shows a perspective view from the side of a fifth exemplary embodiment of an insulation tube according to the present invention,

FIG. 8 schematically shows a cross section of an exemplary embodiment for an electric line according to the present invention, and

FIG. 9 schematically shows a view from the side of the electric line according to FIG. 8.

### DETAILED DESCRIPTION

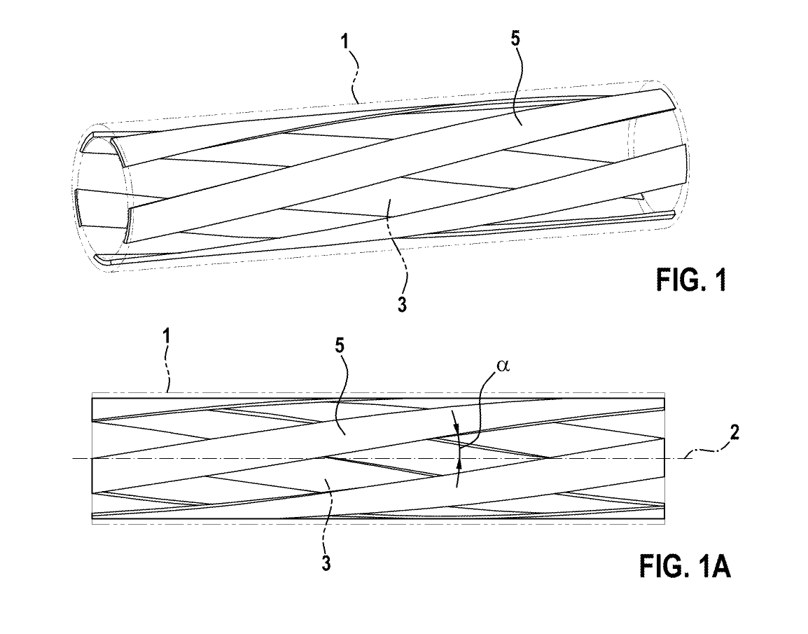

The first exemplary embodiment, illustrated in FIGS. 1 and 1A, of an insulation tube 1 according to the invention comprises a matrix 3, for example, made of silicone, in which web-like elements 5 are embedded. The web-like elements 5, for example, made of polyimide, are formed as strip-shaped webs which extend over the entire axial length of the insulation tube 1 and run helically around the periphery of the insulation tube or on relation to the longitudinal axis 2 of the insulation tube 1. Here, the web-like elements 5 are arranged in the region of the inner wall of the insulation tube 1 and form a part of this inner wall. A web-like element 5 of this type runs at an angle cc of at least 10° with respect to the longitudinal axis 2 of the insulation tube 1. The web-like elements are not interconnected and are arranged side-by-side in the matrix 3 in a manner distributed over the entire periphery.

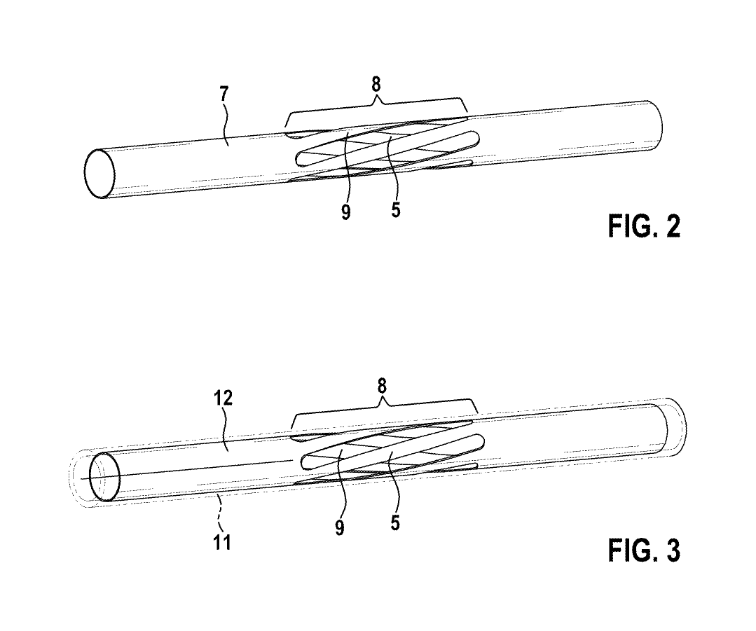

To produce an insulation tube of this type illustrate in FIGS. 1 and 1A, a first thin-walled tubing 7 (see FIG. 2) is first manufactured from a rigid thermoplastic material, for example, polyimide, into which axial openings/slits 9 running in a spiraled manner are cut and run at an angle of at least 10° to the longitudinal axis. These slits 9 are formed in the first tubing 7 in the longitudinal direction along a central portion 8, such that the webs 5 produced as a result are kept in shape by the end portions of the first tubing 7, said end portions not being provided with slits. The state once the first tubing 7 has been slitted is shown in FIG. 2.

The first tubing 7, made of rigid material, is now placed with a core wire (not illustrated) in an injection mold (not illustrated) and is over-molded by a resilient material, for example, silicone, as is shown in FIG. 3. The over-molded resilient material is provided with the reference sign 11, and the second tubing produced after the over-molding process is provided with the reference sign 12. Provided the material of the first tubing 7 and of the over-molding material 11 (here polyimide and silicone, for example) do not enter into an adhesive connection, this arrangement at least ensures a form fit, which contributes to the transmission of tensile forces. The state after the over-molding process is illustrated in FIG. 3.

After the over-molding process, the ends of the second tubing 12 are each cut off, such that the insulation tube 1 is formed merely by the central portion 8, in which the slits were previously formed in the rigid material of the first tubing 7. The insulation tube shown in FIGS. 1 and 1A is thus produced.

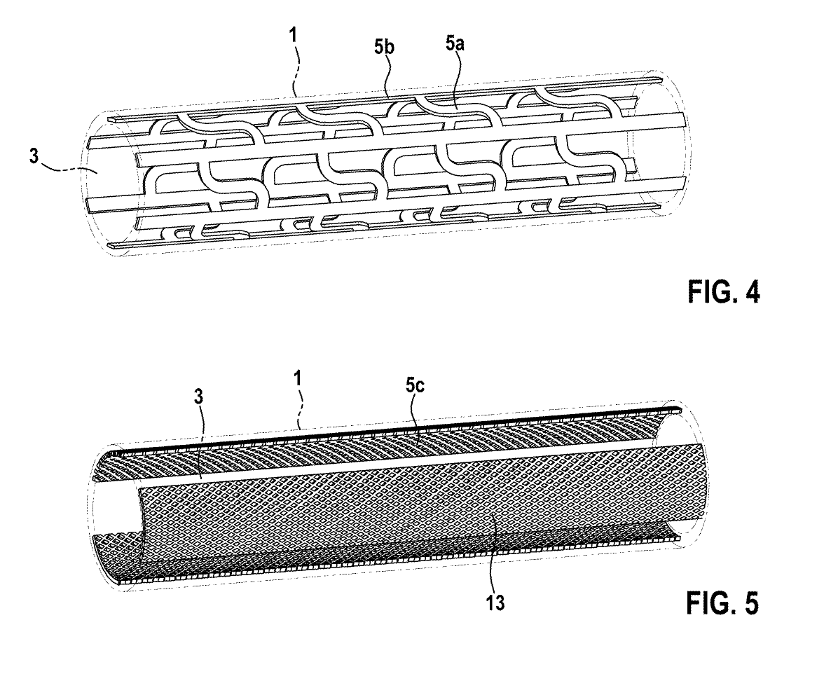

FIG. 4 shows a further exemplary embodiment of an insulation tube 1 according to the present invention, in which the web-like elements formed of the rigid plastic material are formed in part as S-shaped elements 5a and straight elements 5b running in the axial direction, these elements being interconnected. The S-shaped elements 5a run at least in portions along a helix line, and these are connected to the straight elements 5b.

The further exemplary embodiment illustrated in FIG. 5 of an insulation tube 1 according to the present invention comprises web-like elements 5c which are embedded in the resilient matrix 3 and which each run on the whole in the direction of the longitudinal axis 2 of the insulation tube 1, but consist of mesh webs running at an incline with respect to the longitudinal axis or running helically, said webs forming the rhomboid apertures 13.

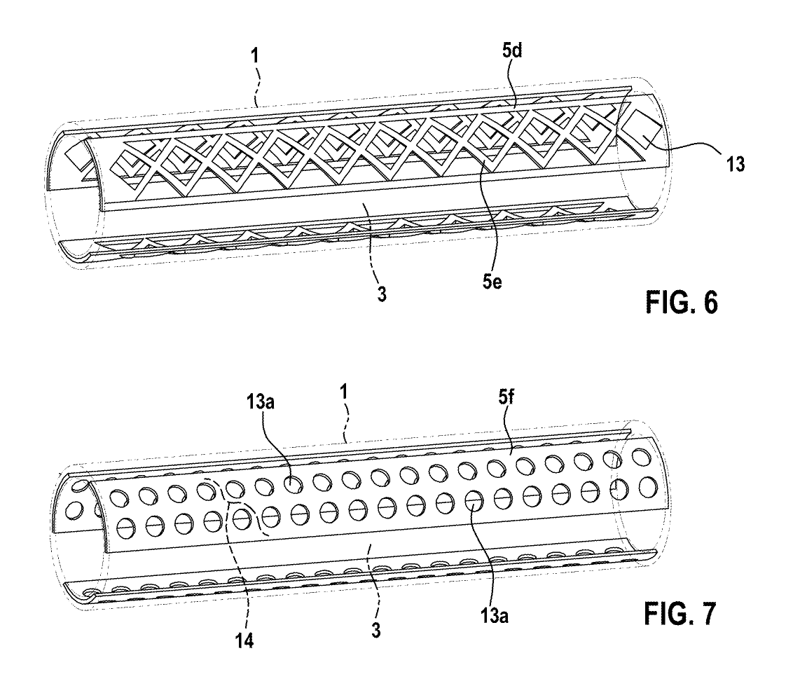

The strip-shaped elements of the further exemplary embodiment illustrated in FIG. 6 are formed very similarly to the exemplary embodiment illustrated in FIG. 5 and are composed at the edge from straight elements 5d running in the axial direction and elements 5e running at an incline with respect to the longitudinal axis of the insulation tube 1, or running helically and forming a mesh, wherein the straight elements 5d and the helical elements 5e are interconnected. Rhomboid apertures 13 are likewise produced by the elements 5e running at an incline.

A further possibility for producing the web-like elements can be inferred from the exemplary embodiment illustrated in FIG. 7, wherein the web-like elements 5f run parallel to the longitudinal axis of the insulation tube 1 and have two rows of circular apertures 13a. Regions 14 which run in portions along a helix line (see the dashed line in FIG. 7 with the reference sign 14) are thus formed in the web-like elements 5f.

With the insulation tubes according to the present invention illustrated in FIGS. 1-1A and 4 to 7, it is possible, with a very thin-walled embodiment, to transmit high tensile forces without losing the advantage in so doing of a possible extensibility in the radial direction. An assembly of the insulation tube on larger component parts is thus possible.

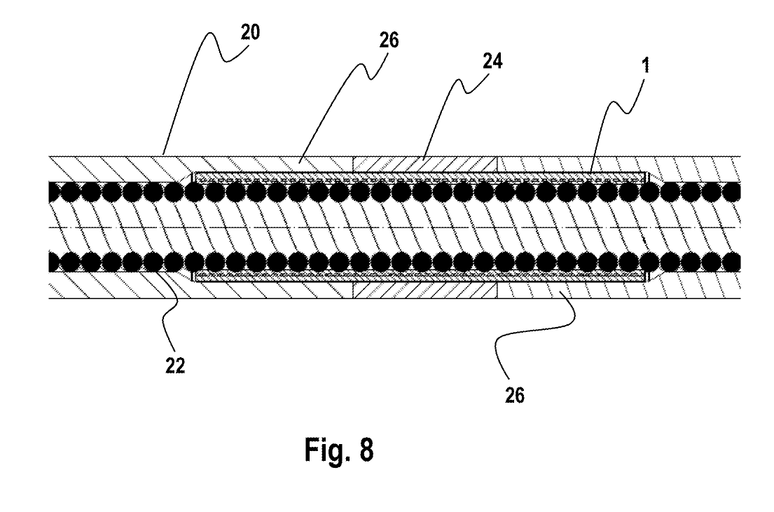

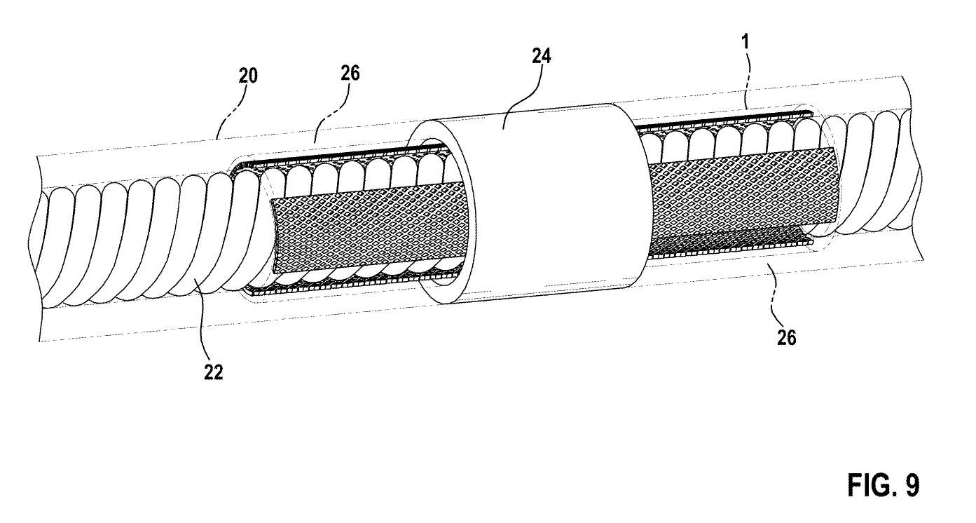

An electric line 20 according to the present invention is illustrated in FIGS. 8 and 9 in the form of a cardiac pacemaker electrode. The electric line 20 has an inner electrode feed line (coil) 22, which creates the electric connection between the electrodes and the electronic components of the cardiac pacemaker. In the region of a ring electrode 24, which is arranged in such a way that it forms part of the outer surface of the electric line, an insulation tube 1 according to FIGS. 1 and 1A is provided and is arranged between the electrode feed line 22 and the ring electrode 24. The length of the insulation tube 1 is approximately 1.5 to 5 times the length of the ring electrode 24, wherein the length of the insulation tube 1 and ring electrode 24 is measured in each case in the direction of the longitudinal axis 2. An insulating sleeve 26 is arranged beside the ring electrode 24 and above the electrode feed line 22 or the insulation tube 1 and can be formed as an insulation tube according to the present invention or is fabricated merely from silicone or another flexible insulating material.

A co-radial coil is used as an electrode feed line. All wires are insulated individually and there are different poles within a coil. For contacting, one of these coil wires is interrupted (the number of wires to be contacted can be varied in accordance with the type of application and number of coil wires) and runs out from the coil composite in a straight line. The insulation layer is removed at this protruding wire. The insulation tube 1 according to the present invention is assembled over this coil region, where the protruding coil wire is guided through. A slitted electrically conductive sleeve 26 with a central outer radial groove is positioned over this. This sleeve 26 is plastically deformed by being pressed together and is thus fixed on the insulation tube and the coil so as to be prevented from slipping. The stripped wire end is wound externally over the ring sleeve into the radial groove. The end of the deposited wire is welded in this position for electric contacting. The outer, actual ring sleeve 24 is then slid over the module and is welded.

The electric line 20 according to the present invention illustrated in FIGS. 8 and 9 is therefore advantageous since, in order to guide outwardly an electric contact of the electrode feed line 22 in the form of a ring electrode 24, the insulating sleeve 26 arranged on the electrode feed line 22 has to be interrupted. This insulation tube is usually deposited on ring heels and adhesively bonded. Due to the small diameter and resultant small adhesive areas, it is often not possible to ensure sufficient tensile force transmission at this point. The insulation tube 1 according to the present invention replaces ring heels that are otherwise conventional and provides the following advantages:

1. The length can be selected such that a sufficiently large adhesive area is provided with the sleeve 26.

2. The rigid region of a ring electrode 24 is thus not lengthened. This improves the navigation during the implantation of the electrode in narrow and acutely angled target vein portions.

3. Due to the use of two identical materials, it is possible to achieve very good adhesive bond strength and therefore tensile force transmission and insulation (for example, the sleeve 26 consists of silicone and the insulation tube 1 consists of the matrix material silicone, the sleeve 26 and the insulation tube 1 being connected by means of silicone adhesives).

The insulation tube 1 according to the present invention therefore passes between the electrode feed line 22 and the ring electrode 24 and, thus, generates a transmission of force from one side to the other side of the ring electrode 24. The insulation tube 1 according to the present invention is flexible, but only needs to have very thin walls. If the insulation tube 1 according to the present invention and the ring electrode 24 are assembled, the sleeve 26, which is assembled along the entire electric line, can be adhesively bonded onto the ends of the insulation tube 1 according to the present invention.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

### LIST OF REFERENCE NUMERALS

1 insulation tube
2 longitudinal axis
3 matrix
5 web-like element
5a, 5b, 5c web-like element
5d, 5e, 5f web-like element
7 first tubing
8 middle portion of the first tube 7
9 slit
11 over-molding
12 second tubing
13, 13a aperture
14 region of the web-like element 5f
20 electric line (cardiac pacemaker electrode)
22 electrode feed line (coil)
24 ring electrode
26 sleeve

I/we claim:

1. An insulation tube for an electric line for medical use, in particular a cardiac pacemaker electrode line, defibrillator electrode line, electric line for nerve stimulation, electric line in catheters, or the like, which comprises:
a matrix consisting of resilient material; and
web-like elements consisting of rigid plastic material and embedded in the matrix, wherein the web-like elements run at least in portions along a helix line which forms an angle of at least 10° in relation to a longitudinal axis of the insulation tube.

2. The insulation tube as claimed in claim 1, wherein the web-like elements are provided separately from one another in the insulation tube.

3. The insulation tube as claimed in claim 1, wherein a plurality of web-like elements extends over the entire axial length of the insulation tube.

4. The insulation tube as claimed in claim 1, wherein the web-like elements have apertures.

5. The insulation tube as claimed in claim 1, wherein the web-like elements are S-shaped.

6. The insulation tube as claimed in claim 1, wherein the web-like elements have a thickness of 0.15 mm at most.

7. The insulation tube as claimed in claim 1, wherein the web-like elements comprise a rigid thermoplastic material.

8. The insulation tube as claimed in claim 7, wherein the rigid thermoplastic material comprises a compound from the group containing polyimide, polyurethane, polyamide, PTFE (polytetrafluoroethylene).

9. The insulation tube as claimed in claim 1, wherein the matrix consists of a resilient plastic.

10. The insulation tube as claimed in claim 9, wherein the resilient plastic comprises a compound from the group containing silicones, rubber and thermoplastic elastomers (TPEs), wherein the TPE comprises a polyether block amide.

11. An electric line for medical use, in particular a cardiac pacemaker electrode line, defibrillator electrode line, electric line for nerve stimulation, electric line in catheters, or the like, comprising:
an insulation tube as claimed in claim 1, wherein the insulation tube is arranged in the region of a ring electrode and/or an electrode tip.

12. The electric line as claimed in claim 11, wherein said line comprises an inflatable balloon.

13. A method for producing an insulation tube for an electric line for medical use, said method comprising the following steps:
fabricating a first thin-walled tubing from a rigid plastic material comprising a rigid thermoplastic material;
introducing openings into the rigid plastic material over part of the length in a central portion of the first tubing, wherein the openings define web-like elements in such a way that the web-like elements run at least in portions along a helix line which forms an angle of at least 10° in relation to a longitudinal axis of the tubing;
embedding the first tubing in a matrix consisting of a resilient material, such that a second tubing is formed which is composed of the first tubing and the surrounding matrix; and
cutting off the ends of the second tubing in such a way that, of the first tubing, merely at least part of the central portion remains and forms the insulation tube.

14. The production method as claimed in claim 13, wherein to embed the first tubing in the matrix formed of the resilient material, the first tubing is over-molded with the material of the matrix.

15. A method for producing an insulation tube for an electric line for medical use, wherein the insulation tube comprises a matrix consisting of resilient material and web-like elements consisting of rigid plastic material and embedded in the matrix, wherein a tubing is first formed from the resilient material by means of extrusion and the web-like elements are then embedded in the extruded material of the matrix, which is not yet fully cured.

16. The method as claimed in claim 15, wherein once the web-like elements have been embedded in the extruded material of the matrix, the intermediate product thus produced is twisted, where applicable with additional heat supply, in such a way that the web-like elements run at least in portions along a helix line, such that this forms an angle of at least 10° in relation to the longitudinal axis of the insulation tube.
